(19) United States
(12) Patent Application Publication
(10) Pub. No.: US 2020/0308240 A1
(43) Pub. Date: Oct. 1, 2020

(54) Targeting Innate Immune Signaling in Neuroinflammation and Neurodegeneration

(71) Applicant: The General Hospital Corporation, Boston, MA (US)

(72) Inventors: Mark William Albers, Winchester, MA (US); Steven Rodriguez, Chelsea, MA (US); Artem Sokolov, Boston, MA (US); Peter Sorger, Winchester, MA (US)

(21) Appl. No.: 16/089,797

(22) PCT Filed: Apr. 3, 2017
(86) PCT No.: PCT/US17/25765

§ 371 Date: Sep. 28, 2018

Related U.S. Application Data
(60) Provisional application No. 62/317,233, filed on Apr. 1, 2016.

(51) Int. Cl. C07K 14/47; A61P 25/28

(52) U.S. Cl. CPC C07K 14/4703; A61K 38/00; A61P 25/28

(57) ABSTRACT

Methods for treating neurodegenerative diseases that can include targeting TANK Binding Kinase 1 (TBK1), I kappa B kinase (IKK), Signal Transducer and Activator of Transcription 1 (STAT1), or Janus Kinase 1 or Janus Kinase 2 (Jak1/2).

### CLAIM OF PRIORITY

This application claims the benefit of U.S. Provisional Patent Application Ser. No. 62/317,233, filed on Apr. 1, 2016. The entire contents of the foregoing are hereby incorporated by reference.

### FEDERALLY SPONSORED RESEARCH OR DEVELOPMENT

This invention was made with Government support under Grant Nos. DP2 OD-006662, R21 NSNS094861, and R34 AG049647 awarded by the National Institutes of Health. The Government has certain rights in the invention.

### TECHNICAL FIELD

Methods for treating neurodegeneration and neuroinflammation that include modulating neuron-intrinsic innate immune signaling pathways, e.g., by targeting TANK Binding Kinase 1 (TBK1), I kappa B kinase (IKK), Signal Transducer and Activator of Transcription 1 (STAT1), or Janus Kinase 1 or Janus Kinase 2 (Jak1/2).

### BACKGROUND

Neurodegenerative diseases, including Alzheimer's disease, have become public health crises (annual cost in USA >$200 billion/year) as the population ages with no known therapies that slow their devastating course.

### SUMMARY

Described herein is the identification and validation of inhibitors (NK-6, NK-7, NK-8, NK-9, NK-10) and their cognate targets as well as components in their signaling pathway that leads to neurodegeneration. Interference with one or more of these validated targets by these inhibitors may prevent neurodegeneration in Alzheimer's disease, amyotrophic lateral sclerosis (ALS), frontotemporal dementia (FTD), viral mediated encephalitidies, and other neurodegenerative diseases in vivo that are triggered by long (>34 nt), cytoplasmic dsRNA.

Thus, described herein are methods for treating a neurodegenerative disease in a subject in need thereof. The methods include administering to the subject a therapeutically effective amount of one or more inhibitors of a protein or transcript listed in table A, e.g., TANK Binding Kinase 1 (TBK1), I kappa B kinase (IKK), Signal Transducer and Activator of Transcription 1 (STAT1), and/or Janus Kinase 1 or Janus Kinase 2 (Jak1/2) protein, e.g., one or more of:
a small molecule inhibitor of a protein listed in Table A;
an inhibitory nucleic acid that targets a transcript listed in Table A; and/or
an antibody that binds to and inhibits a protein listed in Table A;
thereby treating the neurodegenerative disease in the subject. As used herein, Jak1/2 refers to either of Jak1 or Jak2.

Also provided herein are compositions comprising one or more of:
a small molecule inhibitor of a protein listed in Table A;
an inhibitory nucleic acid that targets a transcript listed in Table A; and/or
an antibody that binds to and inhibits a protein listed in Table A, for use in treating a neurodegenerative disease in a subject.

In some embodiments, the neurodegenerative disease is Alzheimer's disease, amyotrophic lateral sclerosis, frontotemporal dementia, Cockayne Syndrome (CS), Xeroderma Pigmentosum (XP), Trichothiodystrophy (TTD), Ataxia with Occulomotor Apraxia-1 (AOA1), Spinocerebellar Ataxia with Axonal Neuropathy (SCAN1), Ataxia Telangiectasia (A-T) or A-T Like Disease (ATLD), ATR-Seckel Syndrome, Nijmegen Breakage Syndrome (NBS), LIG4 Syndrome, Aicardi-Goutier's syndrome and related interferonopathies, Down's Syndrome, or XLF Syndrome.

In some embodiments, the amyotrophic lateral sclerosis (ALS) is C9orf72-linked ALS, fused in sarcoma (FUS)-linked ALS, TAR DNA-binding protein 43 (TDP-43)-linked ALS, C9orf72-linked frontotemporal dementia (FTD), FUS-linked FTD, and TDP-43-linked FTD, C9orf72-linked AD, or sporadic ALS.

In some embodiments, the small molecule inhibitor is selected from the group consisting of BX-795; TPCA-1; Ruxolitinib; fludarabine; and amlexanox.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-H. Neurons in ALS-C9orf72 cases contain high levels of dsRNA in the frontal cortex. A-F) Representative images of immunocytochemistry on sections from the superior frontal cortex (B8,9) with the J2 antibody that recognizes dsRNA. DsRNA positive staining in brown (DAB) can be regularly seen in ALS-C9orf72 (C,F), but not regularly in ALS-SOD (B,E), or control sections (A,D). Insets of digitally magnified cells show the granularity of dsRNA staining in ALS-C9orf72. G) Quantification of the percentage of cells (with nuclear counter stain) that are dsRNA positive as recorded using Fiji in ALS-C9orf72 (n=3) compared to control brains (n=3, p<0.00001), and ALS-SOD brains compared to controls (n=3, p=0.24). H) Quantification of the proteins level of PKR, a double-stranded RNA binding protein, shows it is more highly expressed in ALS-C9orf72 relative to controls (p<0.05).

FIGS. 2A-D. Neurons in ALS-C9orf72 cases contain high levels of dsRNA in the dentate nuclei of the cerebellar cortex. A-B) Representative images of immunocytochemistry on cerebellar sections using the J2 antibody that recognizes dsRNA. DsRNA positive staining in brown (DAB) can be regularly seen in the neurons of the dentate nuclei in ALS-C9orf72 (A), but is not detected at high levels in controls (B). C) Isotype control showing specificity of the J2 antibody. D) Low power image (5×) of the cerebellum showing the restriction of J2 staining to the dentate nuclei-inset shows 40× magnification of neurons in (D) and shows the granularity of J2 staining within these neurons.

FIGS. 3A-G. dsRNA induces a Type I interferon response and is sufficient to induced neurodegeneration in human neurons. A-E) Representative transmitted light images of differentiated ReNVM neurons transfected with increasing doses of PolyIC, a dsRNA mimetic, showing a dose-dependent loss of neurons after 48 hours. F) Quantification of the percentage of viable ReNVM neurons that have been transfected with PolyIC showing a dose-dependent loss in cell viability as measured using Celltiter-Glo. G) Western blots showing the elevated protein expression of type I interferon-stimulated genes in differentiated ReNVM cells 24 hour post transfection with PolyIC, using indicated antibodies.

FIGS. 4A-N. Inhibitors of key type I interferon regulators rescues PolyIC induced neurodegeneration in ReNVM cells. TBK phosphorylation is upregulated by the treatment of PolyIC after 24 hours (first two samples are controls and second two are PolyIC treated) (A). Treatment with inhibitors TPCA-1 (B), BX795 (C), Ruxolitinib (D), Amlexanox (E), Pacritinib-1 (JAK2) (F), XL019 (JAK2) (G), Filgotinib (JAK1) (H), AZD1480 (JAK 1,2) (I), CYT387 (Momelotinib; JAK 1.2) (J), Baricitinib (JAK 1,2) (K), Tofacitinib (JAK1, JAK2, JAK3) (L), IKK16 (IKK 1,2) (M), and TG101348 (JAK2) (N) rescued PolyIC induced neurodegeneration; n.s.=not significant, * P<0.05, * * P<0.01, * ** P<0.001, **** P<0.0001.

FIGS. 5A-C. J2 immunohistochemistry detects higher levels of dsRNA in AD brains with TDP-43 inclusions (A, brown DAB stain) than controls (B), quantified in (C), n=4 AD and n=3 controls, p=0.026).

FIGS. 6A-C. The J2 antibody is specific to dsRNA. A,B) Representative images of sections from the superior frontal cortex (B8,9) with (A) the J2 antibody or (B) the J2 antibody pre incubated with PolyIC (a dsRNA mimetic) showing that the immunocytochemical staining with J2 is specific to dsRNA. C) A dot blot of dsDNA and polyIC probed with the J2 antibody showing the specificity of J2 for dsRNA.

FIG. 7. Clustering solutions of the Mount Sinai Brain Bank dataset for patients with the low (left) and high (right) Braak score. The x and y axes in each plot correspond to individual samples in the dataset. The shade of grey in each cell in the heatmap represents Pearson correlation between mRNA expression profiles of interferome-stimulated gene for the corresponding pair of samples. Complete-linkage hierarchical clustering was applied to the dataset using this correlation as the similarity metric.

### DETAILED DESCRIPTION

Recently, a new mechanistic paradigm for neurodegeneration in the mouse was discovered based on the expression of double stranded RNA (greater than 34 nucleotides) arising from inversions introduced into the genomic sequence through routine nonhomologous end joining repair and other mechanism that cause chromothripsis, a dramatic fracturing and repair of chromosomes (see PCT/US2015/060401). As described herein, genes and gene products have been identified and validated that constitute two related signaling neuroinflammatory pathways triggered by dsRNA by binding to the pattern recognition receptors MDA5, RIG-I, TLR3, Eif2ak2 (PKR) and downstream signaling molecules p38 (MAPK14), Stat1, and HSP27 in the cytoplasm that mediate neurodegeneration and alterations in gene expression programs in neurons in vivo; and demonstrated that these signaling pathways are induced in human brains from patients with pathologically proven Alzheimer's disease, frontotemporal dementia, and amyotrophic lateral sclerosis.

Building on those findings, a novel in vitro human neuronal culture system was developed to recapitulate this pathophysiologic process. These cells can be used, e.g., to identify compounds that modulate dsRNA-mediated neurodegeneration, as a methodology for screening and validation of hits for the novel targets and related targets on the pathway for neurodegeneration and Alzheimer's disease pathogenesis as well as for purposes of drug screening and validation with the primary outcome being loss of neurons. Briefly, a dsRNA-mimetic was introduced into a human neural progenitor immortalized cell line, ReN VM cells, in vitro. There was profound induction of the Type I interferon signaling pathways that were stimulated in mouse models of neurodegeneration including the pattern recognition receptors, PKR and MDA5. Importantly, accelerated neuronal death was also observed over 48 hours that was dependent on the dose of the dsRNA introduced into the cytoplasm. These cell-based phenotypes in human neurons recapitulated and extended findings (described in PCT/US2015/060401) outlining cytoplasmic double-stranded RNA mediated neuronal cell death in multiple transgenic mouse lines (Nd1, Nd2, and Nd3), each expressing a different gene, and a model of induced cytoplasmic dsRNA of GFP causing accelerated neurodegeneration.

Deploying this cell-based in vitro model of accelerated neurodegeneration of human neurons, a number of inhibitors of Type I interferon signaling pathways were tested. A dose-dependent rescue of dsRNA-mediated neurodegeneration was found with four compounds referred to herein as NK-6 (also known as BX-795; presumed target is TBK1), NK-7 (TPCA-1; presumed target is IKK), NK-8 (Ruxolitinib; presumed target is Jak1/2) and NK-9 (fludarabine; presumed target is Stat1); NK-10 (amlexanox; presumed target is TBK1). Surprisingly, other compounds (e.g., PH 797804, which is a p38 inhibitor; C16, which is a PKR inhibitor; and 7DG, which is also a Double-stranded RNA-activated Protein Kinase) that ostensibly inhibiting the targets in related signaling pathways in ReN cells did not show rescue of the neuronal death phenotype.

In addition, expression of cytoplasmic dsRNA was found in the brains of patients with Alzheimer's disease and TDP-43 inclusions and in patients with amyotrophic lateral sclerosis associated with a C9orf72 hexanucleotide expansion with pathology extending to frontotemporal regions (all with TDP-43 inclusions). This result indicates that the mechanisms described herein may be relevant for sporadic Alzheimer's disease with TDP-43 inclusions, which comprises about 20% of disease burden, as well as other neurodegenerative diseases, including sporadic (80% of ALS cases have TDP-43 inclusions) as well as C9orf72—associated frontotemporal dementia and amyotrophic lateral sclerosis (the genomic lesion that is most common to date for both ALS and FTD). Two of the cases with expansion of hexanucleotide repeats at the C9orf7 2 locus was clinically diagnosed with Alzheimer's disease and has Alzheimer's pathology (an apparent paradox that is easily accommodated by this model of neurodegeneration). Moreover, this mechanism implicates genomic lesions that are position independent (non-Mendelian) and thus, would not be revealed by genome wide association studies, since the lesion in each individual is unique. There is clear evidence that the burden of DNA damage is increased in AD and ALS. Targeted sequencing of brain harboring the type I interferon expression signature will defines the nature and origin of cytoplasmic dsRNA sequencing (e.g., genomic lesions, derepressed intrinsic repeats in the genome, e.g., transposons, LINE, or SINE elements, or infectious agents). Moreover, these pathways and their identified inhibitors and future inhibitors based on these targets and/or chemical structures may prevent neurodegeneration related to viral encephalitides, including the profound neurodegeneration observed as microencephaly linked to Zika virus in developing humans. Furthermore, cytoplasmic DNA-triggered innate immune signaling in the CNS, including neurons, glia, and microglia, can activate similar innate immune signaling pathways via cGAS and STING to elicit cell death, including neurons. (1, 2)

Based on these results, the present disclosure provides the identification and validation of inhibitors (NK-6, NK-7, NK-8, NK-9, NK-10) and their cognate targets as well as components in their signaling pathway that leads to neurodegeneration. These cognate targets (TBK1, IKK, STAT1, Jak1/2; see Table A) are gene products traditionally associated with the innate immune system, but they are expressed in the nervous system—often in neurons. In addition, while these drugs have been studied in other disease contexts, neurodegenerative diseases are new therapeutic spaces for these molecules. Interference with one or more of these validated targets by these inhibitors may prevent neurodegeneration in Alzheimer's disease, ALS, viral mediated encephalitidies, and other neurodegenerative diseases in vivo that are triggered by long (>34 nt), cytoplasmic dsRNA.

TABLE A

| Exemplary | | | |
|---|---|---|---|
| Inhibitor* | Target | Human mRNA | Human Protein |
| NK-6 | TBK1 | NM_013254.3 | NP_037386.1 |
| (BX-795) | | | |
| NK-10 | | | |
| (amlexanox) | | | |
| NK-7 | IKK-2 | NM_001190720.2 | NP_001177649.1 |
| (TPCA-1) | | variant 2 | isoform 2 |
| | | NM_001242778.1 | NP_001229707.1 |
| | | variant 7 | isoform 5 |
| | | NM_001556.2 | NP_001547.1 |
| | | variant 1 | isoform 1 |
| NK-8 | Jak1 | NM_002227.2 | NP_002218.2 |
| (Ruxolitinib) | Jak2 | NM_004972.3 | NP_004963.1 |
| NK-9 | Stat1 | NM_007315.3 | NP_009330.1 |
| (fludarabine) | | variant alpha | isoform alpha |
| | | NM_139266.2 | NP_644671.1 |
| | | variant beta | Isoform beta |

*Additional inhibitors are known in the art, including those listed herein.

Methods of Treatment

Based on the work described herein, the present disclosure provides methods for treating neurodegenerative diseases that include administering therapeutically effective amounts of one or more inhibitors of a gene listed in Table A, e.g., TBK1, STAT1, IKK, and/or Jak1/2, e.g., NK-6, NK-7, NK-8, NK-9, and/or NK-10, thereby targeting components of the type 1 interferon dsRNA STAT response signaling pathways that lead to neurodegeneration.

The methods described herein include methods for the treatment of disorders associated with neurodegeneration, e.g., neurodegeneration associated with TDP-43 inclusions. These inclusions can be seen in post-mortem examinations of the brain using immunohistochemistry with an antibody against TDP-43 or phospho-TDP-43, or in CSF or skin samples (fibroblasts) from patients (Jung et al. J Neurol 2014; 261:1344-1348; Pare et al., Acta Neuropathol Commun. 2015; 3:5; Suzuki et al., Acta Neurol Scand. 2010 November; 122(5):367-72); see also Geser et al., Journal of Neurology 2009, 256(8):1205-1214. In some embodiments, the disorder is Alzheimer's disease (e.g., with TDP-43 inclusions), amyotrophic lateral sclerosis (e.g., C9orf72-linked ALS), frontotemporal dementia, Cockayne Syndrome (CS), Xeroderma Pigmentosum (XP), Trichothiodystrophy (TTD), Ataxia with Occulomotor Apraxia-1 (AOA1), Spinocerebellar Ataxia with Axonal Neuropathy (SCAN1), Ataxia Telangiectasia (A-T) or A-T Like Disease (ATLD), ATR-Seckel Syndrome, Nijmegen Breakage Syndrome (NBS), LIG4 Syndrome, Aicardi-Goutier's syndrome and related interferonopathies, Down's Syndrome (Davidson et al., Acta Neuropathol. 2011 December; 122(6):703-13), or XLF Syndrome. In some embodiments, the methods include administering a therapeutically effective amount of one or more inhibitors of TBK1, IKK, and/or Jak1/2, e.g., NK-6, NK-7, NK-8, and/or NK-10.

In some embodiments, the disease is associated with a viral encephalitides, e.g., the profound neurodegeneration observed after Zika virus (ZIKV) infection, particularly microcephaly in fetuses born to mothers infected with ZIKV (Rubin et al., N Engl J Med 374:984-985 (2016); Mlakar et al., N Engl J Med 374:951-958 (2016)). Zika virus enhances transcription of the MDAS gene and is sensitive to the antiviral effects of type I interferons, indicating triggering of a signaling program similar to that described herein in mice and cells (see, e.g., Hamel et al., J Virol 89:8880-8896 (2015)).

In some embodiments, the methods include administering a therapeutically effective amount of one or more inhibitors of TBK1, STAT1, IKK, and/or Jak1/2, e.g., NK-6, NK-7, NK-8, NK-9, and/or NK-10 to a subject who is in need of, or who has been determined to be in need of, such treatment.

As used in this context, to “treat” means to ameliorate at least one symptom or sign of the disorder associated with neurodegeneration, e.g., improvement in trajectory of psychometric testing, reduction or delay/slowing of brain atrophy on MRI, reversal of hypometabolism on PET imaging, decreased progression of or amyloid or tau pathology on PET imaging. Often, neurodegeneration results in a loss of cognitive, sensory, or physical (motor) function; thus, a treatment can result in a reduction in rate or severity of loss of cognitive, sensory or physical (motor) function and a return or approach to normal cognitive, sensory or physical (motor) function, or preservation of cognitive or physical function. Administration of a therapeutically effective amount of a compound described herein for the treatment of a condition associated with neurodegeneration will result in decreased neuronal cell death.

Inhibitors of TBK1, IKK, Jak1/2, and Stat1

One of the targets of NK-6 (BX-795) and NK-10 (amlexanox) is TBK1; both NK-6 and NK-10 inhibit the kinase activity of TBK1. Mutations in TBK1 are associated with familial ALS, and the presumed mechanism is loss of the kinase function of TBK1 as a dominant loss of function mechanism (Freischmidt et al., Nat. Neurosci. 18: 631-636 (2015); Cirulli et al., Science 347: 1436-1441 (2015); Pottier et al., Acta Neuropathol. 130: 77-92 (2015). Hasan et al., J Immunol 195: 4573-4577 (2015) proposed inhibiting TBK1 with a 6-aminopyrazolopyrimidine derivative known as Compound II as an effective treatment for TREX1-associated autoimmune diseases and potentially other interferonopathies, but Brenner et al. stated that “Long-term pharmacological TBK1 inhibition, as proposed [by Hasan et al.] for the treatment of autoimmune diseases (citing Hasan et al. and Yu et al., Nat. Commun. 6: 6074 (2015)), could thus pose a risk of causing ALS or FTD.” (Brenner et al., J Immunol. 2016 Jan. 15; 196(2):530-1). The present data is inconsistent with the model of Brenner et al. In contrast, and without wishing to be bound by theory, it is proposed that the mutations of TBK1 may disrupt the precise localization of TBK1 by disrupting its interactions, perhaps through altering polyubiquitination of TBK1 or the interaction with polyubiquitinated TBK1 with other proteins, including an adapter protein optineurin, which is also a validated gene known to cause ALS and FTD (see, e.g., Sakaguchi et al., Neurosci Lett 2011; 505:279-281; Bury et al. Oligogenic inheritance of optineurin (OPTN) and C9ORF72 mutations in ALS highlights localisation of OPTN in the TDP-43-negative inclusions of C9ORF72-ALS. Neuropathology 2015; doi: 10.1111/neup.12240; Li et al., Amyotroph Lateral Scler Frontotemporal Degener 2015; 16:485-489), enabling TBK1 to ectopically phosphorylate substrates in a dysregulated fashion that cause premature neuronal death. Thus, inhibition of expression or activity, e.g., kinase activity, of TBK1 can be used therapeutically as described herein, e.g., to treat ALS, Alzheimer's disease, FTD, and other neurodegenerative diseases, including viral encephalitides with dysregulated TBK1 and/or TDP-43 inclusions. TBK1 inhibitors can include small molecules (e.g., NK-6 (BX-795), NK-10 (amlexanox), and Compound II, structure shown below, see Ou et al., Mol Cell. 2011 Feb. 18; 41(4):458-70) and biologic agents such as anti-TBK1 antibodies and inhibitory nucleic acids that bind to and inhibit TBK1 to reduce the activity of TBK1. In some embodiments the methods using TBK1 inhibitors do not include their use to treat Aicardi-Goutieres disease.

Similarly, inhibitors of IKK can also be used therapeutically as described herein, e.g., to treat ALS, Alzheimer's disease, Aicardi-Goutieres disease, FTD, and other neurodegenerative diseases, including viral encephalitides with dysregulated IKK and/or TDP-43 inclusions. IKK inhibitors can include small molecules (e.g., NK-7 (2-[(Aminocarbonyl)amino]-5-(4-fluorophenyl)-3-thiophenecarboxamide, also known as TPCA-1), IKK16 (N-(4-Pyrrolidin-1-yl-piperidin-1-yl)-[4-(4-benzo[b]thiophen-2-yl-pyrimidin-2-ylamino)phenyl]carboxamide hydrochloride, which targets IKK 1,2), and biologic agents such as anti-IKK antibodies and inhibitory nucleic acids that bind to and inhibit IKK to reduce the activity of IKK.

Inhibitors of JAK1/JAK2 can also be used therapeutically as described herein, e.g., to treat ALS, Alzheimer's disease, Aicardi-Goutieres disease, FTD, and other neurodegenerative diseases, including viral encephalitides with dysregulated JAK1/JAK2 and/or TDP-43 inclusions. JAK1/JAK2 inhibitors can include small molecules (e.g., NK-8 (Ruxolitinib), Pacritinib-1 (11-(2-pyrrolidin-1-yl-ethoxy)-14,19-dioxa-5,7,26-triaza-tetracyclo[19.3.1.1(2,6).1(8,12)]heptacosa-1(25),2(26),3,5,8,10,12(27),16,21,23-decaene, targets JAK2), XL019 ((S)-N-(4-(2-((4-morpholinophenyl)amino)pyrimidin-4-yl)phenyl)pyrrolidine-2-carboxamide, targets JAK2), Filgotinib (N-[5-[4-[(1,1-Dioxido-4-thiomorpholinyl)methyl]phenyl][1,2,4]triazolo[1,5-a]pyridin-2-yl]cyclopropanecarboxamide, targets JAK1), AZD1480 (5-Chloro-N2-[(1S)-1-(5-fluoro-2-pyrimidinyl)ethyl]-N4-(5-methyl-1H-pyrazol-3-yl)-2,4-pyrimidinediamine, targets JAK 1,2), CYT387 (N-(Cyanomethyl)-4-[2-[[4-(4-morpholinyl)phenyl]amino]-4-pyrimidinyl]benzamide, also known as Momelotinib; targets JAK 1,2), Baricitinib (2-(3-(4-(7H-pyrrolo[2,3-d]pyrimidin-4-yl)-1H-pyrazol-1-yl)-1-(ethylsulfonyl)azetidin-3-yl)acetonitrile, targets JAK 1,2), Tofacitinib (3-((3R,4R)-4-methyl-3-(methyl(7H-pyrrolo [2,3-d]pyrimidin-4-yl)amino)piperidin-1-yl)-3-oxopropanenitrile, targets JAK1, JAK2, JAK3), and TG101348 (2.N-tert-butyl-3-(5-methyl-2-(4-(2-(pyrrolidin-1-yl)ethoxy)phenylamino) pyrimidin-4-ylamino)benzenesulfonamide, targets JAK2)), and biologic agents such as anti-JAK1/JAK2 antibodies and inhibitory nucleic acids that bind to and inhibit JAK1/JAK2 to reduce the activity of JAK1/JAK2.

Inhibitors of Stat1 can also be used therapeutically as described herein, e.g., to treat ALS, Alzheimer's disease, Aicardi-Goutieres disease, FTD, and other neurodegenerative diseases, including viral encephalitides with dysregulated Stat1 and/or TDP-43 inclusions. Stat1 inhibitors can include small molecules (e.g., NK-9 (fludarabine)) and biologic agents such as anti-Stat1 antibodies and inhibitory nucleic acids that bind to and inhibit Stat1 to reduce the activity of Stat1. Small molecule inhibitors of Stat1 include Fludarabine, NSC 74589, SD 1008, Stattic, and WP1066. Inhibitors of MapKapK-2 include NQD1, HI TOPK 032; PF 3644022; (10r)-10-methyl-3-(6-methylpyridin-3-yl)-9,10,11,12-tetrahydro-8h-[1,4]diazepino[5′,6′:4,5]thieno[3,2-f]quinolin-8-one; (3r)-3-(aminomethyl)-9-methoxy-1,2,3,4-tetrahydro-5h-[1]benzothieno[3,2-e][1,4]diazepin-5-one; (4r)-n-[4-({[2-(dimethylamino)ethyl]amino}carbonyl)-1,3-thiazol-2-yl]-4-methyl-1-oxo-2,3,4,9-tetrahydro-1h-beta-carboline-6-carboxamide; 2-(2-quinolin-3-ylpyridin-4-yl)-1,5,6,7-tetrahydro-4h-pyrrolo[3,2-c]pyridin-4-one; 2-[2-(2-fluorophenyl)pyridin-4-yl]-1,5,6,7-tetrahydro-4h-pyrrolo[3,2-c]pyridin-4-one; and 3-{[(1r)-1-phenylethyl]amino}-4-(pyridin-4-ylamino)cyclobut-3-ene-1,2-dione. Inhibitors of MapKapK-3 include DB08358 (2-(2-quinolin-3-ylpyridin-4-yl)-1,5,6,7-tetrahydro-4h-pyrrolo[3,2-c]pyridin-4-one) and DB07728 (2-[2-(2-fluorophenyl)pyridin-4-yl]-1,5,6,7-tetrahydro-4h-pyrrolo[3,2-c]pyridin-4-one).

The small molecules useful in the present methods can be synthesized using methods known in the art or obtained from commercial sources, e.g., Tocris or other source, e.g., as listed in the DrugBank database.

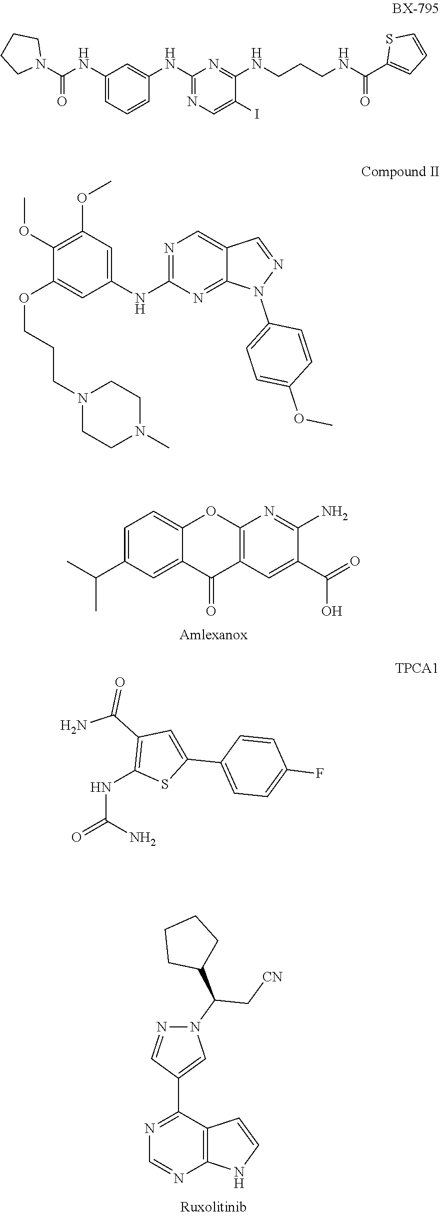

Inhibiting type 1 interferon Signaling with Sequence-Specific Oligonucleotides

As described herein, oligonucleotides (“oligos”) that hybridize specifically to a gene or transcript listed in Table A can be used to cause a reduction in neuronal cell death in response to dsRNA. Exemplary target sequences are listed in Table A.

In some embodiments, the oligos hybridize to at least 7, 8, 9, 10, 11, 12, 13, 14, 15, 16, 17, 18, 19, 20, 21, 22, 23, 24, 25, 26, 27, 28, 29, 30 or more consecutive nucleotides of the target sequence.

In some embodiments, the methods include introducing into the cell an oligo that specifically binds, or is complementary, to a gene listed in Table A. A nucleic acid that “specifically” binds primarily to the target, i.e., to a gene listed in Table A RNA but not to other non-target RNAs. The specificity of the nucleic acid interaction thus refers to its function (e.g., inhibiting a gene listed in Table A) rather than its hybridization capacity. Oligos may exhibit nonspecific binding to other sites in the genome or other mRNAs, without interfering with binding of other regulatory proteins and without causing degradation of the non-specifically-bound RNA. Thus this nonspecific binding does not significantly affect function of other non-target RNAs and results in no significant adverse effects. These methods can be used to treat a subject, e.g., a subject with cancer, by administering to the subject a composition (e.g., as described herein) comprising an oligo that binds to a gene listed in Table A. Examples of oligos and target sequences are provided herein.

As used herein, treating includes “prophylactic treatment” which means reducing the incidence of or preventing (or reducing risk of) a sign or symptom of a disease in a patient at risk for the disease, and “therapeutic treatment”, which means reducing signs or symptoms of a disease, reducing progression of a disease, reducing severity of a disease, in a patient diagnosed with the disease.

In some embodiments, the methods described herein include administering a composition, e.g., a sterile composition, comprising an oligo that is complementary to a gene listed in Table A sequence as described herein. Oligos for use in practicing the methods described herein can be an antisense or small interfering RNA, including but not limited to an shRNA or siRNA. In some embodiments, the oligo is a modified nucleic acid polymer (e.g., a locked nucleic acid (LNA) molecule), a gapmer, or a mixmer.

Oligos have been employed as therapeutic moieties in the treatment of disease states in animals, including humans. Oligos can be useful therapeutic modalities that can be configured to be useful in treatment regimens for the treatment of cells, tissues and animals, especially humans.

For therapeutics, an animal, preferably a human, suspected of having cancer is treated by administering an oligo in accordance with this disclosure. For example, in one non-limiting embodiment, the methods comprise the step of administering to the animal in need of treatment a therapeutically effective amount of an oligo as described herein.

### Oligonucleotides

Oligos useful in the present methods and compositions include antisense oligonucleotides, ribozymes, external guide sequence (EGS) oligonucleotides, siRNA compounds, single- or double-stranded RNA interference (RNAi) compounds such as siRNA compounds, molecules comprising modified bases, locked nucleic acid molecules (LNA molecules), antagomirs, peptide nucleic acid molecules (PNA molecules), mixmers, gapmers, and other oligomeric compounds or oligonucleotide mimetics that hybridize to at least a portion of a gene listed in Table A and modulate its function. In some embodiments, the oligos include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., WO2010/040112. However, in some embodiments the oligo is not an miRNA, an stRNA, an shRNA, an siRNA, an RNAi, or a dsRNA.

In some embodiments, the oligos are 10 to 50, 13 to 50, or 13 to 30 nucleotides in length. One having ordinary skill in the art will appreciate that this embodies oligonucleotides having antisense (complementary) portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. It is understood that non-complementary bases may be included in such oligos; for example, an oligo 30 nucleotides in length may have a portion of 15 bases that is complementary to the targeted gene listed in Table A RNA. In some embodiments, the oligonucleotides are 15 nucleotides in length. In some embodiments, the antisense or oligonucleotide compounds of the invention are 12 or 13 to 30 nucleotides in length. One having ordinary skill in the art will appreciate that this embodies oligos having antisense (complementary) portions of 12, 13, 14, 15, 16, 17, 18, 19, 20, 21, 22, 23, 24, 25, 26, 27, 28, 29 or 30 nucleotides in length, or any range therewithin.

Preferably the oligo comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the oligos are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric oligos of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the oligo comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH,˜N(CH₃)˜O˜CH₂ (known as a methylene(methylimino) or MMI backbone], CH₂—O—N (CH₃)—CH₂, CH₂—N (CH₃)—N (CH₃)—CH₂ and O—N (CH₃)—CH₂—CH₂ backbones, wherein the native phosphodiester backbone is represented as O—P—O—CH,); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5, 177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596, 086; 5,602,240; 5,610,289; 5,602,240; 5,608,046; 5,610,289; 5,618,704; 5,623, 070; 5,663,312; 5,633,360; 5,677,437; and 5,677,439, each of which is herein incorporated by reference.

Modified oligonucleotides are also known that include oligonucleotides that are based on or constructed from arabinonucleotide or modified arabinonucleotide residues. Arabinonucleosides are stereoisomers of ribonucleosides, differing only in the configuration at the 2′-position of the sugar ring. In some embodiments, a 2′-arabino modification is 2′-F arabino. In some embodiments, the modified oligonucleotide is 2′-fluoro-D-arabinonucleic acid (FANA) (as described in, for example, Lon et al., Biochem., 41:3457-3467, 2002 and Min et al., Bioorg. Med. Chem. Lett., 12:2651-2654, 2002; the disclosures of which are incorporated herein by reference in their entireties). Similar modifications can also be made at other positions on the sugar, particularly the 3′ position of the sugar on a 3′ terminal nucleoside or in 2′-5′ linked oligonucleotides and the 5′ position of 5′ terminal nucleotide.

PCT Publication No. WO 99/67378 discloses arabinonucleic acids (ANA) oligomers and their analogues for improved sequence specific inhibition of gene expression via association to complementary messenger RNA.

Other preferred modifications include ethylene-bridged nucleic acids (ENAs) (e.g., International Patent Publication No. WO 2005/042777, Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties). Preferred ENAs include, but are not limited to, 2′-O,4′-C-ethylene-bridged nucleic acids.

Examples of LNAs are described in WO 2008/043753 and WO2007031091 and include compounds of the following formula.

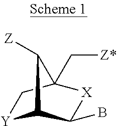

where X and Y are independently selected among the groups —O—, —S—, —N(H)—, N(R)—, —CH₂— or —CH— (if part of a double bond), —CH₂—O—, —CH₂—S—, —CH₂—N(H)—, —CH₂—N(R)—, —CH₂—CH₂— or —CH₂—CH— (if part of a double bond), —CH═CH—, where R is selected from hydrogen and C₁₋₄-alkyl; Z and Z* are independently selected among an internucleoside linkage, a terminal group or a protecting group; B constitutes a natural or non-natural nucleotide base moiety; and the asymmetric groups may be found in either orientation.

Preferably, the LNA used in the oligomer of the invention comprises at least one LNA unit according any of the formulas

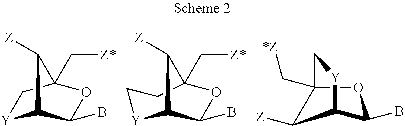

wherein Y is —O—, —S—, —NH—, or N(R^{H}); Z and Z* are independently selected among an internucleoside linkage, a terminal group or a protecting group; B constitutes a natural or non-natural nucleotide base moiety, and RH is selected from hydrogen and C₁₋₄-alkyl.

Preferably, the Locked Nucleic Acid (LNA) used in an oligomeric compound, such as an antisense oligonucleotide, as described herein comprises at least one nucleotide comprises a Locked Nucleic Acid (LNA) unit according any of the formulas shown in Scheme 2 of PCT/DK2006/000512 (WO2007031091).

Preferably, the LNA used in the oligomer of the invention comprises internucleoside linkages selected from —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected from hydrogen and C₁₋₄-alkyl.

Specifically preferred LNA units are shown in scheme 3:

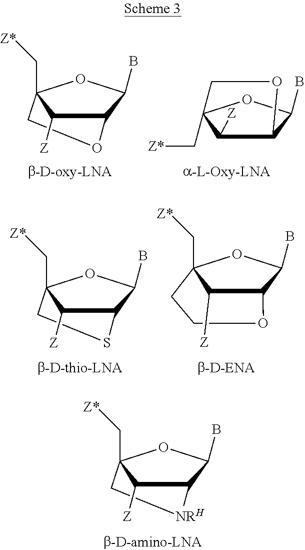

The term “thio-LNA” comprises a locked nucleotide in which at least one of X or Y in the general formula above is selected from S or —CH₂—S—. Thio-LNA can be in both beta-D and alpha-L-configuration.

The term “amino-LNA” comprises a locked nucleotide in which at least one of X or Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ena-LNA” comprises a locked nucleotide in which Y in the general formula above is —CH₂—O— (where the oxygen atom of —CH₂—O— is attached to the 2′-position relative to the base B).

LNAs are described in additional detail below. One or more substituted sugar moieties can also be included, e.g., one of the following at the 2′ position: OH, SH, SCH₃, F, OCN, OCH₃ OCH₃, OCH₃ O(CH₂)n CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH_{3;} SO2 CH_{3;} ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-0-CH₂CH₂OCH₃, also known as 2′-O-(2-methoxyethyl)] (Martin et al, HeIv. Chim. Acta, 1995, 78, 486). Other preferred modifications include 2′-methoxy (2′-0-CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligos can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl)adenine, 6-aminopurine, 2-aminopurine, 2-chloro-6-aminopurine and 2,6-diaminopurine or other diaminopurines. See, e.g., Kornberg, “DNA Replication,” W. H. Freeman & Co., San Francisco, 1980, pp75-77; and Gebeyehu, G., et al. Nucl. Acids Res., 15:4513 (1987)). A “universal” base known in the art, e.g., inosine, can also be included. 5-Me-C substitutions have been shown to increase nucleic acid duplex stability by 0.6-1.2<0>C. (Sanghvi, in Crooke, and Lebleu, eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the modifications described herein may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Oligos can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in “The Concise Encyclopedia of Polymer Science And Engineering”, pages 858-859, Kroschwitz, ed. John Wiley & Sons, 1990; those disclosed by Englisch et al., Angewandle Chemie, International Edition, 1991, 30, page 613, and those disclosed by Sanghvi, Chapter 15, Antisense Research and Applications,” pages 289-302, Crooke, and Lebleu, eds., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2° C. (Sanghvi, et al., eds, “Antisense Research and Applications,” CRC Press, Boca Raton, 1993, pp. 276-278) and are presently preferred base substitutions, even more particularly when combined with 2′-O-methoxyethyl sugar modifications. Modified nucleobases are described in U.S. Pat. Nos. 3,687,808, as well as 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the oligos are chemically linked to one or more moieties or conjugates that enhance the activity, cellular distribution, or cellular uptake of the oligonucleotide. For example, one or more oligos, of the same or different types, can be conjugated to each other; or oligos can be conjugated to targeting moieties with enhanced specificity for a cell type or tissue type. Such moieties include, but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

The oligos useful in the present methods are sufficiently complementary to the target a gene listed in Table A, e.g., hybridize sufficiently well and with sufficient biological functional specificity, to give the desired effect. “Complementary” refers to the capacity for pairing, through base stacking and specific hydrogen bonding, between two sequences comprising naturally or non-naturally occurring (e.g., modified as described above) bases (nucleosides) or analogs thereof. For example, if a base at one position of an oligo is capable of hydrogen bonding with a base at the corresponding position of a gene listed in Table A, then the bases are considered to be complementary to each other at that position. 100% complementarity is not required. As noted above, oligos can comprise universal bases, or inert abasic spacers that provide no positive or negative contribution to hydrogen bonding Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and SantaLucia, Nucl. Acids Research, 2005; 33 (19): 6258-6267.

In some embodiments, the location on a target a gene listed in Table A binding site to which an oligo hybridizes is a region to which a protein binding partner binds. The identification of these binding sites is described in the Examples below. Routine methods can be used to design an oligo that binds to a selected strong or moderate binding site sequence with sufficient specificity. In some embodiments, the methods include using bioinformatics methods known in the art to identify regions of secondary structure, e.g., one, two, or more stem-loop structures, or pseudoknots, and selecting those regions to target with an oligo. For example, methods of designing oligonucleotides similar to the oligos described herein, and various options for modified chemistries or formats, are exemplified in Lennox and Behlke, Gene Therapy (2011) 18: 1111-1120, which is incorporated herein by reference in its entirety, with the understanding that the inhibitory oligonucleotides of the present disclosure do not target miRNA ‘seed regions’.

While the specific sequences of certain exemplary target segments are set forth herein, one of skill in the art will recognize that these serve to illustrate and describe particular embodiments within the scope of the present invention. Additional target segments are readily identifiable by one having ordinary skill in the art in view of this disclosure. One having skill in the art armed with the sequences provided herein will be able, without undue experimentation, to identify further preferred regions to target with complementary oligos.

In the context of the present disclosure, hybridization means base stacking and hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as the term is used in the art, refers to the capacity for precise pairing between two nucleotides. For example, if a nucleotide at a certain position of an oligonucleotide is capable of hydrogen bonding with a nucleotide at the same position of a gene listed in Table A molecule, then the oligo and the a gene listed in Table A molecule are considered to be complementary to each other at that position. The oligos and the a gene listed in Table A molecule are complementary to each other when a sufficient number of corresponding positions in each molecule are occupied by nucleotides that can hydrogen bond with each other through their bases. Thus, “specifically hybridizable” and “complementary” are terms which are used to indicate a sufficient degree of complementarity or precise pairing such that stable and specific binding occurs between the oligo and the a gene listed in Table A molecule. For example, if a base at one position of an oligo is capable of hydrogen bonding with a base at the corresponding position of a gene listed in Table A, then the bases are considered to be complementary to each other at that position. 100% complementarity is not required.

It is understood in the art that a complementary nucleic acid sequence need not be 100% complementary to that of its target nucleic acid to be specifically hybridizable. A complementary nucleic acid sequence for purposes of the present methods is specifically hybridizable when binding of the sequence to the target a gene listed in Table A molecule interferes with the normal function of a gene listed in Table A to cause a loss of activity and there is a sufficient degree of complementarity to avoid non-specific binding of the sequence to non-target sequences under conditions in which avoidance of the non-specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the oligos useful in the methods described herein have at least 80% sequence complementarity to a target region within the target nucleic acid, e.g., 90%, 95%, or 100% sequence complementarity to the target region within a gene listed in Table A. For example, an antisense compound in which 18 of 20 nucleobases of the antisense oligonucleotide are complementary, and would therefore specifically hybridize, to a target region would represent 90 percent complementarity. Percent complementarity of an oligo with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Antisense and other compounds of the invention that hybridize to a gene listed in Table A are identified through routine experimentation. In general the oligos must retain specificity for their target, i.e., either do not directly bind to, or do not directly significantly affect expression levels of, transcripts other than the intended target.

Target-specific effects, with corresponding target-specific functional biological effects, are possible even when the oligo exhibits non-specific binding to a large number of non-target RNAs. For example, short 8 base long oligos that are fully complementary to a gene listed in Table A may have multiple 100% matches to hundreds of sequences in the genome, yet may produce target-specific effects, e.g. upregulation of a specific target gene through inhibition of a gene listed in Table A activity. 8-base oligos have been reported to prevent exon skipping with with a high degree of specificity and reduced off-target effect. See Singh et al., RNA Biol., 2009; 6(3): 341-350. 8-base oligos have been reported to interfere with miRNA activity without significant off-target effects. See Obad et al., Nature Genetics, 2011; 43: 371-378.

For further disclosure regarding oligos, please see US2010/0317718 (antisense oligos); US2010/0249052 (double-stranded ribonucleic acid (dsRNA)); US2009/0181914 and US2010/0234451 (LNA molecules); US2007/0191294 (siRNA analogues); US2008/0249039 (modified siRNA); and WO2010/129746 and WO2010/040112 (oligos).

Antisense

In some embodiments, the oligos are antisense oligonucleotides. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to a gene listed in Table A in vitro, and are expected to inhibit the activity of a gene listed in Table A in vivo. Thus, oligonucleotides are chosen that are sufficiently complementary to the target, i.e., that hybridize sufficiently well and with sufficient biological functional specificity, to give the desired effect.

### Modified Bases, Including Locked Nucleic Acids (LNAs)

In some embodiments, the oligos used in the methods described herein comprise one or more modified bonds or bases. Modified bases include phosphorothioate, methylphosphonate, peptide nucleic acids, or locked nucleic acids (LNAs). Oligos that have been modified (locked nucleic acid—LNA) have demonstrated the “on target” specificity of this approach. Preferably, the modified nucleotides are part of locked nucleic acid molecules, including [alpha]-L-LNAs. LNAs include ribonucleic acid analogues wherein the ribose ring is “locked” by a methylene bridge between the 2′-oxygen and the 4′-carbon—i.e., oligonucleotides containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., a gene listed in Table A sequences as described herein.

The modified base/LNA molecules can include molecules comprising, e.g., 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the a gene listed in Table A. The modified base/LNA molecules can be chemically synthesized using methods known in the art.

The modified base/LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of a modified base/LNA molecule; for example, a series of oligonucleotides of 10-30 nucleotides spanning the length of a target gene listed in Table A can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing modified base/LNA molecules are known in the art; for example, LNA sequences will bind very tightly to other LNA sequences, so it is preferable to avoid significant complementarity within an LNA molecule. Contiguous runs of three or more Gs or Cs, or more than four LNA residues, should be avoided where possible (for example, it may not be possible with very short (e.g., about 9-10 nt) oligonucleotides). In some embodiments, the LNAs are xylo-LNAs.

For additional information regarding LNA molecules see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

As demonstrated herein, LNA molecules can be used as a valuable tool to manipulate and aid analysis of a gene listed in Table A RNAs. Advantages offered by an LNA molecule-based system are the relatively low costs, easy delivery, and rapid action. While other oligos may exhibit effects after longer periods of time, LNA molecules exhibit effects that are more rapid, e.g., a comparatively early onset of activity, are fully reversible after a recovery period following the synthesis of new a gene listed in Table A molecules, and occur without causing substantial or substantially complete RNA cleavage or degradation. One or more of these design properties may be desired properties of the oligos of the invention. Additionally, LNA molecules make possible the systematic targeting of domains within much longer nuclear transcripts. The LNA technology enables high-throughput screens for functional analysis of a gene listed in Table A RNAs and also provides a novel tool to manipulate chromatin states in vivo for therapeutic applications.

In various related aspects, the methods described herein include using LNA molecules to target a gene listed in Table A for a number of uses, including as a research tool to probe the function of a gene listed in Table A, e.g., in vitro or in vivo. The methods include selecting one or more desired a gene listed in Table A sequences, designing one or more LNA molecules that target the a gene listed in Table A sequences, providing the designed LNA molecule, and administering the LNA molecule to a cell or animal.

In still other related aspects, the LNA molecules targeting a gene listed in Table A as described herein can be used to create animal or cell models of conditions associated with altered a gene listed in Table A expression.

Antagomirs

In some embodiments, the oligo is an antagomir. Antagomirs are chemically modified antisense oligonucleotides that can target a gene listed in Table A. For example, an antagomir for use in the methods described herein can include a nucleotide sequence sufficiently complementary to hybridize to a gene listed in Table A target sequence of about 12 to 25 nucleotides, preferably about 15 to 23 nucleotides.

In some embodiments, antagomirs include a cholesterol moiety, e.g., at the 3′-end. In some embodiments, antagomirs have various modifications for RNase protection and pharmacologic properties such as enhanced tissue and cellular uptake. For example, in addition to the modifications discussed above for antisense oligos, an antagomir can have one or more of complete or partial 2′-O-methylation of sugar and/or a phosphorothioate backbone. Phosphorothioate modifications provide protection against RNase or other nuclease activity and their lipophilicity contributes to enhanced tissue uptake. In some embodiments, the antagomir cam include six phosphorothioate backbone modifications; two phosphorothioates are located at the 5′-end and four at the 3′-end, but other patterns of phosphorothioate modification are also commonly employed and effective. See, e.g., Krutzfeldt et al., Nature 438, 685-689 (2005); Czech, N Engl J Med 2006; 354:1194-1195 (2006); Robertson et al., Silence. 1:10 (2010); Marquez and McCaffrey, Hum Gene Ther. 19(1):27-38 (2008); van Rooij et al., Circ Res. 103(9):919-928 (2008); and Liu et al., Int. J. Mol. Sci. 9:978-999 (2008). Krutzfeld et al. (2005) describe chemically engineered oligonucleotides, termed ‘antagomirs’, that are reported to be efficient and specific silencers of endogenous miRNAs in mice.

In general, the design of an antagomir avoids target RNA degradation due to the modified sugars present in the molecule. The presence of an unbroken string of unmodified sugars supports RNAseH recruitment and enzymatic activity. Thus, typically the design of an antagomir will include bases that contain modified sugar (e.g., LNA), at the ends or interspersed with natural ribose or deoxyribose nucleobases.

Antagomirs useful in the present methods can also be modified with respect to their length or otherwise the number of nucleotides making up the antagomir. In some embodiments, the antagomirs must retain specificity for their target, i.e., must not directly bind to, or directly significantly affect expression levels of, transcripts other than the intended target. In some embodiments, antagomirs may exhibit nonspecific binding that does not produce significant undesired biologic effect, e.g., the antagomirs do not affect expression levels of non-target transcripts or their association with regulatory proteins or regulatory RNAs.

Interfering RNA, Including siRNA/shRNA

In some embodiments, the oligo sequence that is complementary to a gene listed in Table A can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc Natl Acad Sci USA 99:6047-6052, (2002).

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

Ribozymes

In some embodiments, the oligos are ribozymes. Trans-cleaving enzymatic nucleic acid molecules can also be used; they have shown promise as therapeutic agents for human disease (Usman & McSwiggen, 1995 Ann. Rep. Med. Chem. 30, 285-294; Christoffersen and Man, 1995 J. Med. Chem. 38, 2023-2037). Enzymatic nucleic acid molecules can be designed to cleave specific caRNA targets within the background of cellular RNA. Such a cleavage event renders the caRNA non-functional.

In general, enzymatic nucleic acids with RNA cleaving activity act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R. Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al, 1994, TIBTECH 12, 268; Bartel et al, 1993, Science 261:1411-1418; Szostak, 1993, TIBS 17, 89-93; Kumar et al, 1995, FASEB J., 9, 1183; Breaker, 1996, Curr. Op. Biotech., 1, 442). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 MM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

Making and Using Oligos

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. If desired, nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)).

Preferably, oligos of the invention are synthesized chemically. Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066; WO/2008/043753 and WO/2008/049085, and the references cited therein.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

It is understood that any of the modified chemistries or formats of oligos described herein can be combined with each other, and that one, two, three, four, five, or more different types of modifications can be included within the same molecule.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y. (1993).

### Modification Patterns

In some embodiments, the inhibitory oligonucleotide comprises locked nucleic acids (LNA), ENA modified nucleotides, 2′-O-methyl nucleotides, or 2′-fluoro-deoxyribonucleotides. In some embodiments, the inhibitory oligonucleotide comprises alternating deoxyribonucleotides and 2′-fluoro-deoxyribonucleotides. In some embodiments, the inhibitory oligonucleotide comprises alternating deoxyribonucleotides and 2′-O-methyl nucleotides. In some embodiments, the inhibitory oligonucleotide comprises alternating deoxyribonucleotides and ENA modified nucleotides. In some embodiments, the inhibitory oligonucleotide comprises alternating deoxyribonucleotides and locked nucleic acid nucleotides. In some embodiments, the inhibitory oligonucleotide comprises alternating locked nucleic acid nucleotides and 2′-O-methyl nucleotides.

The oligonucleotide may comprise deoxyribonucleotides flanked by at least one bridged nucleotide (e.g., a LNA nucleotide, cEt nucleotide, ENA nucleotide) on each of the 5′ and 3′ ends of the deoxyribonucleotides. The oligonucleotide may comprise deoxyribonucleotides flanked by 1, 2, 3, 4, 5, 6, 7, 8 or more bridged nucleotides (e.g., LNA nucleotides, cEt nucleotides, ENA nucleotides) on each of the 5′ and 3′ ends of the deoxyribonucleotides. In some embodiments, the 5′ nucleotide of the oligonucleotide is a deoxyribonucleotide. In some embodiments, the 5′ nucleotide of the oligonucleotide is a locked nucleic acid nucleotide. In some embodiments, the nucleotides of the oligonucleotide comprise deoxyribonucleotides flanked by at least one locked nucleic acid nucleotide on each of the 5′ and 3′ ends of the deoxyribonucleotides. In some embodiments, the nucleotide at the 3′ position of the oligonucleotide has a 3′ hydroxyl group or a 3′ thiophosphate.

In some embodiments, the inhibitory oligonucleotide comprises phosphorothioate internucleotide linkages. In some embodiments, the single stranded oligonucleotide comprises phosphorothioate internucleotide linkages between at least two nucleotides. In some embodiments, the single stranded oligonucleotide comprises phosphorothioate internucleotide linkages between all nucleotides.

It should be appreciated that the oligonucleotide can have any combination of modifications as described herein.

As an example, the oligonucleotide may comprise a nucleotide sequence having one or more of the following modification patterns.
(a) (X)Xxxxxx, (X)xXxxxx, (X)xxXxxx, (X)xxxXxx, (X)xxxxXx and (X)xxxxxX,
(b) (X)XXxxxx, (X)XxXxxx, (X)XxxXxx, (X)XxxxXx, (X)XxxxxX, (X)xXXxxx, (X)xXxXxx, (X)xXxxXx, (X)xXxxxX, (X)xxXXxx, (X)xxXxXx, (X)xxXxxX, (X)xxxXXx, (X)xxxXxX and (X)xxxxXX,
(c) (X)XXXxxx, (X)xXXXxx, (X)xxXXXx, (X)xxxXXX, (X)XXxXxx, (X)XXxxXx, (X)XXxxxX, (X)xXXxXx, (X)xXXxxX, (X)xxXXxX, (X)XxXXxx, (X)XxxXXx (X)XxxxXX, (X)xXxXXx, (X)xXxxXX, (X)xxXxXX, (X)xXxXxX and (X)XxXxXx,
(d) (X)xxXXX, (X)xXxXXX, (X)xXXxXX, (X)xXXXxX, (X)xXXXXx, (X)XxxXXXX, (X)XxXxXX, (X)XxXXxX, (X)XxXXx, (X)XXxxXX, (X)XXxXxX, (X)XXxXXx, (X)XXXxxX, (X)XXXxXx, and (X)XXXXxx,
(e) (X)xXXXXX, (X)XxXXXX, (X)XXxXXX, (X)XXXxXX, (X)XXXXxX and (X)XXXXXx, and
(f) XXXXXX, XxXXXXX, XXxXXXX, XXXxXXX, XXXXxXX, XXXXXxX and XXXXXXx, in which “X” denotes a nucleotide analogue, (X) denotes an optional nucleotide analogue, and “x” denotes a DNA or RNA nucleotide unit. Each of the above listed patterns may appear one or more times within an oligonucleotide, alone or in combination with any of the other disclosed modification patterns.

In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012. 22: 344-359; Nowotny et al., Cell, 121:1005-1016, 2005; Kurreck, European Journal of Biochemistry 270: 1628-1644, 2003; FLuiter et al., Mol Biosyst. 5(8):838-43, 2009). In some embodiments, the oligonucleotide is a mixmer (includes alternating short stretches of LNA and DNA; Naguibneva et al., Biomed Pharmacother. 2006 Nov; 60(9):633-8; Orom et al., Gene. 2006 May 10; 3720:137-41).

### Additional Sequence Structural Information

The inhibitory oligonucleotides described herein may have a sequence that does not contain guanosine nucleotide stretches (e.g., 3 or more, 4 or more, 5 or more, 6 or more consecutive guanosine nucleotides). In some embodiments, oligonucleotides having guanosine nucleotide stretches have increased non-specific binding and/or off-target effects, compared with oligonucleotides that do not have guanosine nucleotide stretches.

The inhibitory oligonucleotides have a sequence that has less than a threshold level of sequence identity with every sequence of nucleotides, of equivalent length, that map to a genomic position encompassing or in proximity to an off-target gene. For example, an oligonucleotide may be designed to ensure that it does not have a sequence that maps to genomic positions encompassing or in proximity with all known genes (e.g., all known protein coding genes) other than the gene of interest. The oligonucleotide is expected to have a reduced likelihood of having off-target effects. The threshold level of sequence identity may be 50%, 60%, 70%, 80%, 85%, 90%, 95%, 99% or 100% sequence identity.

The inhibitory oligonucleotides may have a sequence that is complementary to a region that encodes an RNA that forms a secondary structure comprising at least two single stranded loops. In some embodiments, oligonucleotides that are complementary to a region that encodes an RNA that forms a secondary structure comprising one or more single stranded loops (e.g., at least two single stranded loops) have a greater likelihood of being active (e.g., of being capable of activating or enhancing expression of a target gene) than a randomly selected oligonucleotide. In some cases, the secondary structure may comprise a double stranded stem between the at least two single stranded loops. Accordingly, the area of complementarity between the oligonucleotide and the nucleic acid region may be at a location of the PRC2 associated region that encodes at least a portion of at least one of the loops. In some embodiments, the predicted secondary structure RNA (e.g., of the a gene listed in Table A sequence) containing the nucleic acid region is determined using RNA secondary structure prediction algorithms, e.g., RNAfold, mfold. In some embodiments, oligonucleotides are designed to target a region of the RNA that forms a secondary structure comprising one or more single stranded loop (e.g., at least two single stranded loops) structures which may comprise a double stranded stem between the at least two single stranded loops.

The inhibitory oligonucleotide may have a sequence that is has greater than 30% G-C content, greater than 40% G-C content, greater than 50% G-C content, greater than 60% G-C content, greater than 70% G-C content, or greater than 80% G-C content. The inhibitory oligonucleotide may have a sequence that has up to 100% G-C content, up to 95% G-C content, up to 90% G-C content, or up to 80% G-C content.

In some embodiments, the region of complementarity of the inhibitory oligonucleotide is complementary with at least 8 to 15, 8 to 30, 8 to 40, or 10 to 50, or 5 to 50, or 5 to 40 bases, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive nucleotides of a gene listed in Table A as known in the art or disclosed herein. In some embodiments, the region of complementarity is complementary with at least 8, 10, 12, 14, 16, 18, or 20 consecutive nucleotides of a gene listed in Table A as known in the art or disclosed herein.

Antibodies

Antibodies that specifically bind to and inhibit a target listed in Table A can be used disrupt Type 1 interferon signaling and cause reduction in neuronal cell death in response to dsRNA, and therefore be used to treat neurodegenerative diseases as described herein.

The term “antibody” as used herein refers to an immunoglobulin molecule or an antigen-binding portion thereof. Examples of antigen-binding portions of immunoglobulin molecules include F(ab) and F(ab′)₂ fragments, which retain the ability to bind antigen. The antibody can be polyclonal, monoclonal, recombinant, chimeric, de-immunized or humanized, fully human, non-human, (e.g., murine), or single chain antibody. In some embodiments the antibody has effector function and can fix complement. In some embodiments, the antibody has reduced or no ability to bind an Fc receptor. For example, the antibody can be an isotype or subtype, fragment or other mutant, which does not support binding to an Fc receptor, e.g., it has a mutagenized or deleted Fc receptor binding region. Methods for making antibodies and fragments thereof are known in the art, see, e.g., Harlow et. al., editors, Antibodies: A Laboratory Manual (1988); Goding, Monoclonal Antibodies: Principles and Practice, (N.Y. Academic Press 1983); Howard and Kaser, Making and Using Antibodies: A Practical Handbook (CRC Press; 1st edition, Dec. 13, 2006); Kontermann and Dübel, Antibody Engineering Volume 1 (Springer Protocols) (Springer; 2nd ed., May 21, 2010); Lo, Antibody Engineering: Methods and Protocols (Methods in Molecular Biology) (Humana Press; Nov. 10, 2010); and Dübel, Handbook of Therapeutic Antibodies: Technologies, Emerging Developments and Approved Therapeutics, (Wiley-VCH; 1 edition Sep. 7, 2010).

Antibodies that bind to the protein targets listed in Table A and inhibit their activity are known in the art and can be obtained using routine methods from commercial sources.

Methods of Screening

Cell lines with and without knockouts of TBK1, MDA5, PKR, STAT1, JAK1, JAK2, IKK, e.g., created using CRISPR/Cas9 or other routine molecular biological methodology can be used for screening and evaluating the dsRNA mechanism of neurodegeneration using either human (ReN VM and ReN CX) or other mammalian neuronal cell lines, iPS-derived human neurons that are grown in either conventional or three-dimensional culture conditions for screening of small molecules or biologics. The cell line could be derived from the Nd1 and/or Nd2 mouse lines or it could be generated using sense and anti-sense mRNAs from a marker protein, such as but not restricted to, GFP. The GFP/anti sense GFP would be amenable to high throughput screening. We would expect neuronal loss and reduced GFP fluorescence in the baseline state. Compounds that would inhibit this mechanism of neurodegeneration would increase neuronal density and increase GFP fluorescence, two outcomes that are quantifiable in automated fashion using a high content imaging platform.

### EXAMPLES

The invention is further described in the following examples, which do not limit the scope of the invention described in the claims.

### Example 1

### Cytoplasmic Double Stranded RNA, Present in Neurons of C9orf72-Mediated ALS and in Alzheimer's Disease Brain with TDP-43 Inclusions, Triggers Neurodegeneration in Human Neurons

Recent observations by multiple laboratories 7-10 indicate that both sense and antisense RNA strands are produced from the most common genetic lesion associated with amyotrophic lateral sclerosis (ALS) and frontotemporal dementia (FTD), the GGGGCC repeat in the first intron of C9orf72 11, 12. Whether the neurodegeneration associated with C9orf72 is due to RAN dipeptide toxicity 13, 14, RNA toxicity 7, 8, 1115, 16, alterations in nuclear-cytoplasmic transport 17 and/or additional events remains unanswered. Accounting for the Type I neuroinflammatory changes associated with ALS pathogenesis 18, 19, an alternative hypothesis was considered: that long sense and anti-sense RNA expressed from the expanded hexanucleotide repeat of C9orf72 could form cytoplasmic double-stranded RNA (dsRNA) thereby evoking an intrinsic innate immune response leading to neurodegeneration. Indeed, some viral encephalitides result in progressive neurodegeneration in the setting of a fulminant innate immune response to cytoplasmic dsRNA 20, 21, leading to the induction and activation of Type I interferon networks, including the double stranded protein kinase, pattern recognition receptor PKR (EIF2AK2). Activated PKR triggers signaling cascades that ultimately can lead to neuronal death, including loss of neurons following poliovirus infection in mice 22. Type I interferon signaling networks are induced in the choroid plexus of aging mice and negatively impact brain function 23. Interestingly, genetic suppression of PKR results in enhanced cognition in mice 24.

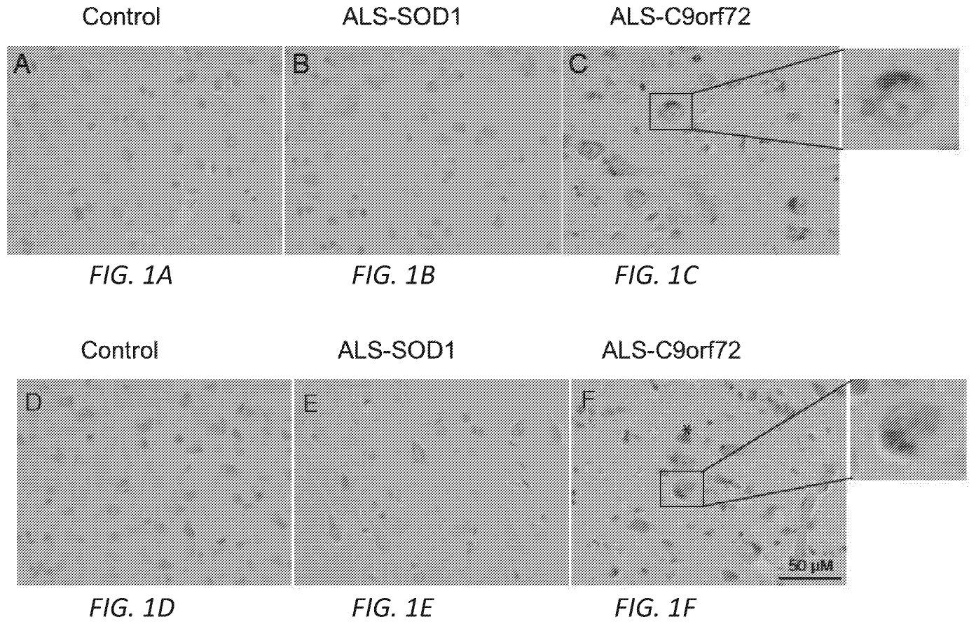

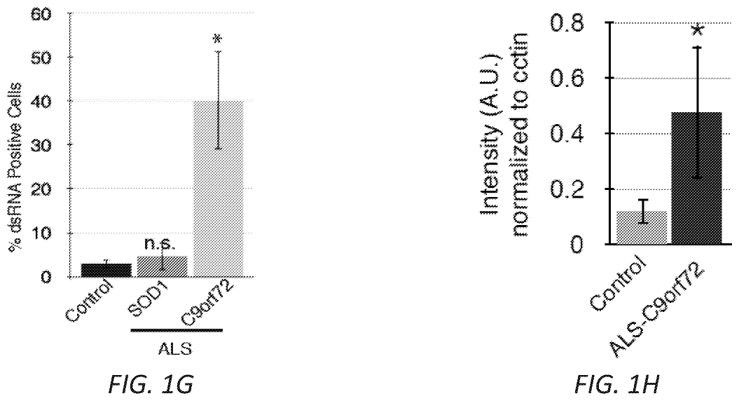

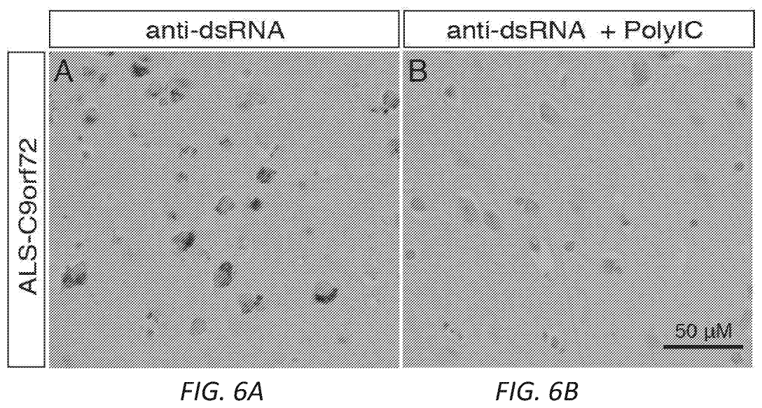

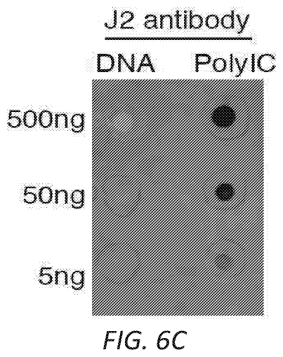

To test this hypothesis, we immunostained frontal cortices from patients clinically and pathologically diagnosed with ALS in the setting of a hexanucleotide expansion of C9orf72 using an antibody that recognizes dsRNA in a sequence-independent fashion 25 This antibody is specific for the dsRNA mimetic polyIC and not DNA(Sup. FIG. 1C). We find that there is a preponderance of cytoplasmic dsRNA in ALS-C9orf72 cases (41%, ±5% SEM, n=3) and one FTD case compared to age-matched controls (10%, ÷4% SEM, n=3; p=0.003) (FIG. 1A-F). The signal was largely abrogated by immunostaining with a isotype primary antibody control and by preincubating the antibody with the dsRNA-mimetic poly-inosine-cytosine (poly IC) (FIGS. 6A and B). The morphology of this specific cytoplasmic staining was typically granular and perinuclear. We found the highest density of cytoplasmic dsRNA in layers 5 of the frontal cortex, whereas TDP-43 inclusions have a higher density in more superficial layers of frontal cortex. Consistent with a pathologic role for the cytoplasmic dsRNA, we also observed a significant increase in protein levels of the cytoplasmic dsRNA receptor PKR in C9orf72 cases relative to the control cases (FIG. 1H).

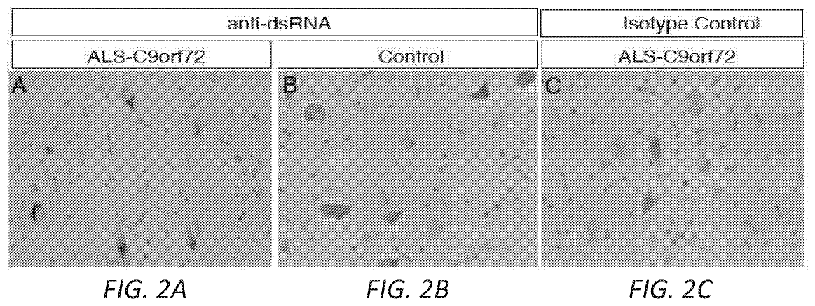

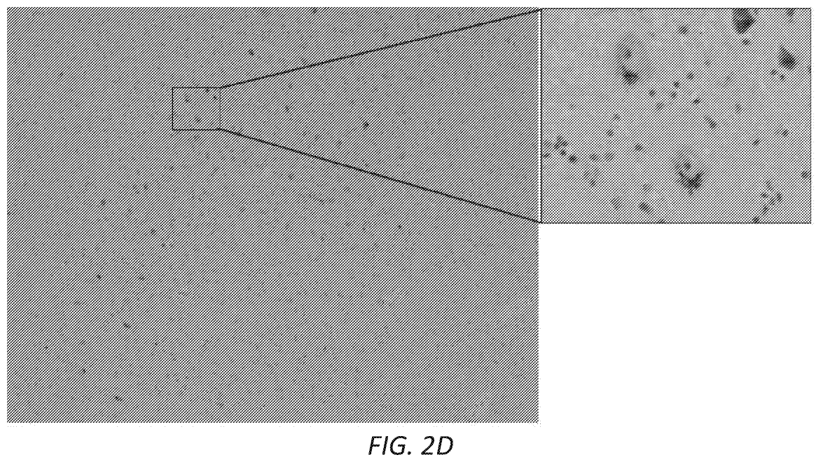

DsRNA cytoplasmic inclusions were also found in neurons in the dentate nuclei in the cerebellum of these ALS cases (FIG. 2A). Immunostaining adjacent sections of human cerebellum with an antibody against the GA dipeptide revealed cytoplasmic inclusions, consistent with a previous report 26, which implicates RNA expression of the hexanucleotide repeat region of C9orf72 in neurons that also harbor cytoplasmic dsRNA. The specific immunostaining did not overlap with lipofuscin. In ALS cases with SOD1 mutations, which do not have TDP-43 pathology, dsRNA cytoplasmic inclusions were not detected in the frontal lobes or in the cerebellum (FIGS. 1B and 1E).

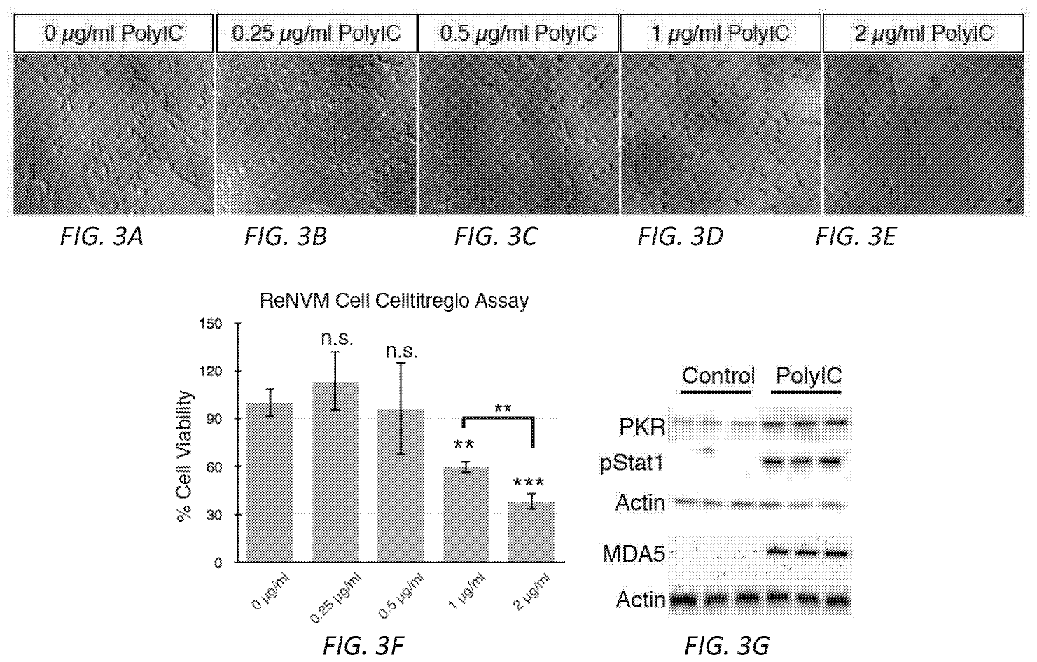

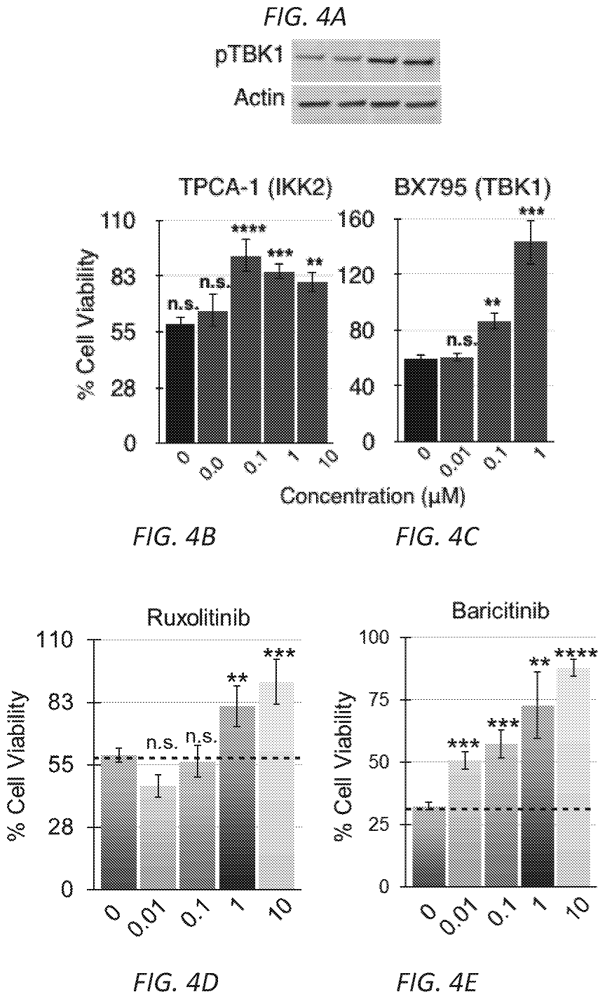

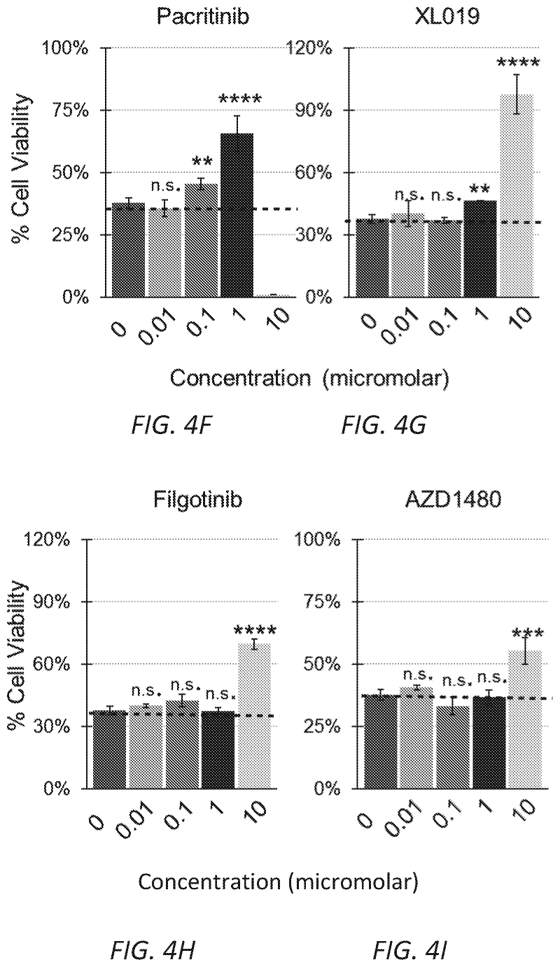

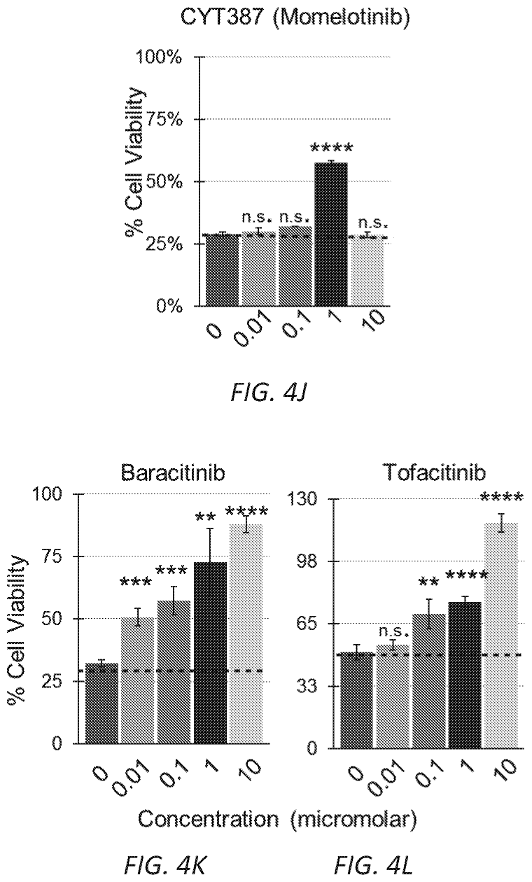

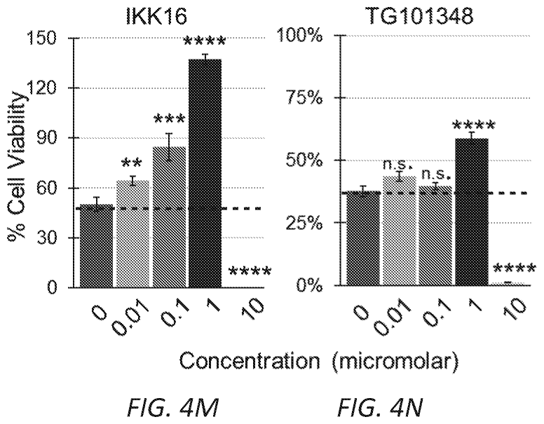

To determine if cytoplasmic dsRNA is sufficient to mediate neurodegeneration in human neurons, we transfected neuronal differentiated ReNcell VM cells 27, 28, a human neural cell line derived from ventral mesencephalon, with escalating doses of poly IC (which mimics a GC dinucleotide repeat). After 2 days in culture, we found a dose-dependent reduction in their viability (FIG. 3A-F) relative to mock-transfected neurons. We observed a marked induction of the pattern recognition receptors MDA5 and PKR that are cytoplasmic receptors for dsRNA (FIG. 3G) and TBK1 phosphorylation (FIG. 4A). In addition, downstream signaling events, i.e., MAVS reduction and IRF3 translocation to the nucleus for the MDA5 pathway, and phosphorylation of p38 and HSP27 for the PKR pathway, indicate that that neurons are capable of mounting this innate immune response intrinsically. Taken together, these results indicate that cytoplasmic dsRNA is sufficient to induce death in a human neurons in vitro. While there is mounting evidence for non-cell autonomous inflammatory changes in astrocytes and microglia in ALS, these results indicate that neurons are capable on intrinsically mounting an inflammatory response, a process that we refer to as neuroninflammation.

Employing a candidate approach to probe the signaling pathways evoked by cytoplasmic dsRNA, we found that the kinase inhibitor BX795 (Feldman et al., J Biol Chem 2005; 280:19867-19874), which targets the TBK1 kinase (kinase-screen.mrc.ac.uk) rescues poly-IC-mediated neurodegeneration in a dose dependent manner (FIG. 4C). Moreover, NK-10 amlexonox, another TBK1 inhibitor also rescued neurodegeneration (FIG. 4E). In addition, TPCA1, a kinase inhibitor that targets the structurally and functional related kinase IKKB 30 (FIG. 4B), and IKK16, which targets IKK 1,2 (FIG. 4M) also reversed poly-IC-mediated neurodegeneration in the setting of reduced IRF3 phosphorylation. Finally, the JAK1 inhibitor Filgotinib (FIG. 4H); the JAK2 inhibitors Pacritinib-1, XL019, and TG101348 (FIGS. 4F, 4G, 4N); the JAK1/JAK2 inhibitors Ruxolitinib, CYT387 (Momelotinib), and Baricitinib (FIGS. 4D, 4J, 4K); and the JAK1/JAK2/JAK3 inhibitor Tofacitinib (FIG. 4L) also blocked neurodegeneration in dose dependent manner. These results establish these proteins and IRF3 phosphorylation as candidates to treat dsRNA-mediated neurodegeneration in C9orf72-associated ALS, where we observe elevated IRF3 levels in the brain regions with detectable cytoplasmic dsRNA. Many mutations in the TBK1 coding region are linked to ALS and FTD in an autosomal dominant manner. Expression of these mutant isoforms have been difficult to detect at the RNA and protein levels, giving rise to the hypothesis that haploinsufficiency of TBK1 drives neurodegeneration. Inhibition of TBK1 kinase activity challenge the hypothesis that LOF mutations cause the pathogenesis. TBK1 is activated by polyubiquitination and regulated by proteins binding to its CCH domain, including optineurin which binds TBK1, is mutated in some familial forms of ALS that abrogates inhibition of IRF3 4.

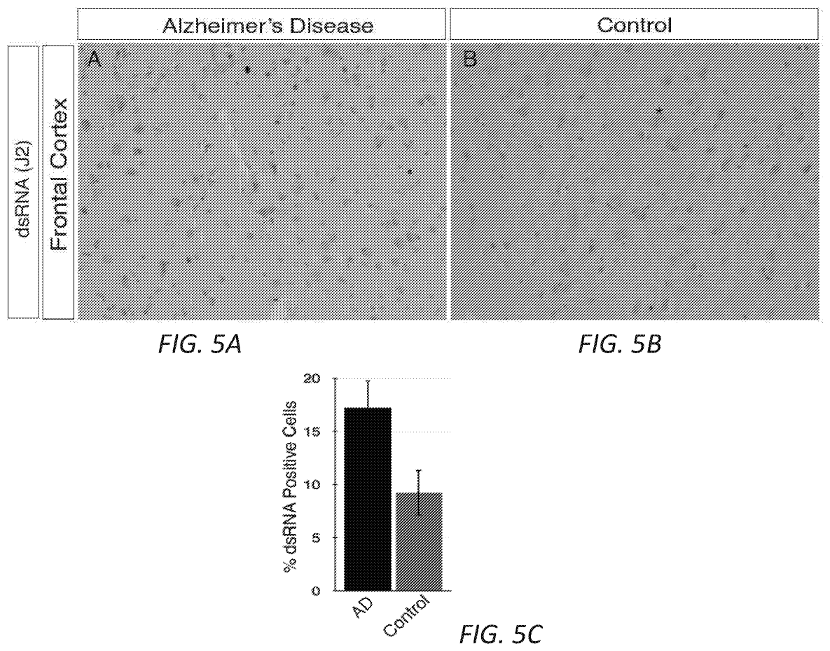

We posit that cytoplasmic dsRNA may trigger induction of MDA5 and PKR in ALS patients with TDP-43 inclusions. Aberrant expression of transposable elements has been shown in FTD patient brains, which can form dsRNA by intra- and inter-molecular interactions 31. Loss of TDP43, a gene linked to ALS whose protein product aggregates and mis-localizes to the cytoplasm from the nucleus in several neurodegenerative diseases, leads to the aberrant accumulation of dsRNA 32 and activation of the type I IFN response in the rodent brain 33. In an unbiased screen FUS was identified as a negative regulator of the type I IFN response 34. Also, there is an aberrant accumulation of sense and antisense RNA from the C9orf72 gene linked to ALS and FTD 7, 8 Both partial loss of function of TDP-43 and overexpression of pathogenic alleles of TDP-43 are associated with the derepression of endogenous retroviral sequences embedded in human genomes 35-37, which may trigger a viral mimicry pathological cascade. Indeed, we found cytoplasmic dsRNA in neurons in the amygdala of cognitively normal individuals with Braak stage I and II tangle deposition as well as demented patients with Braak stage V and VI pathology. Moreover, we found significantly greater cytoplasmic dsRNA inclusions in the neurons of frontal lobes of AD patients with severe dementia relative to the neurons of frontal lobes from individuals who were cognitively normal (FIGS. 5A-C). The frequency of dsRNA staining exceeded the frequency of TDP-43 inclusions, and were present in pen-nuclear granules. AD cases without detectable TDP-43 aggregates were not enriched in cytoplasmic dsRNA inclusions.

We have identified cytoplasmic dsRNA in neurons of subsets of ALS patients with TDP-43 cytoplasmic inclusions. Cytoplasmic dsRNA evokes Type I innate immune signaling 38 and is sufficient to mediate neurodegeneration 39. Possible intrinsic origins of the cytoplasmic dsRNA species in neurons include genomic lesions (e.g. C9orf72 hexanucleotide expansions) 7, genomic inversions (either inherited or acquired somatically as a consequence of aging) 40, derepression of endogenous retroviruses and transposons 35, 37, 41, ectopic splicing 42, and impaired RNA editing 43(optineurin is an ALS gene with long intramolecular dsRNA hairpin in its 3′ UTR that is edited 5, 6). Moreover, non-cell autonomous processes are possible, e.g., propagation of dsRNA from neighboring or synaptically connected cells via exosomes or direct transfer and exogenous viral infection 20. Taken together, our findings demonstrate that dsRNA accumulates in the cytoplasm of neurons in a subset of cases of ALS and AD and that activation of cytoplasmic dsRNA sensors contribute to the rich tapestry of neurodegenerative processes that underlie clinically-classified disease 44. Inhibition of one or more of these innate immune pathways may slow propagated neurodegeneration and offer clinical benefit, particularly if treatment is initiated at a sufficiently early stage.

### Example 2

### Machine Learning Analysis of Publicly Available Accelerated Medicine Partnership

Alzheimer's disease gene expression data implicates a signature gene set of the type I interferon signaling pathway that was discovered in dsRNA-mimetic treated cultured human ReN neurons followed by mass spectrometry analysis of proteins, in both early and late stages of Alzheimer's disease. We analyzed the well-characterized, inflammation-linked JAK-STAT signaling pathway (Mukai et al., Nat Neurosci 11(11): 1302-1310 2008). Using the Mount Sinai Brain Bank (MSBB) dataset from the AMP-AD database (Zhang et al., Cell. 2013 Apr. 25; 153(3):707-20) (dataset: doi:10.7303/syn2580853) and looked at Interferon Stimulated Genes (ISGs). We asked whether interferon-stimulated genes (ISGs), an amplified, downstream marker of JAK-STAT signaling, were enriched in AD samples. All genes in the dataset were ranked based on correlation of their mRNA expression to the Braak score, a neuropathological marker that measures AD progression. We found that a set of canonical ISGs (Schoggins et al., Nature. 2011 Apr. 28; 472(7344):481-5) was significantly enriched (Gene Set Enrichment Analysis (GSEA), p-value=0.0026) in this ranking, which suggests that expression might be linked to disease state. To further evaluate this connection, we looked at cultured human ReN neurons that had been treated with poly(I:C), a synthetic analog of double-stranded RNA that stimulates the innate immune response and promotes neuroinflammation, analogous to that observed in AD brains. By ranking genes based on their protein expression levels (as measured by quantitative protein Mass Spectrometry), we observed significant positive enrichment (GSEA, p-value<0.0001) for ISGs. This result confirms poly(I:C) as an immune response stimulant and activator of JAK-STAT signaling.

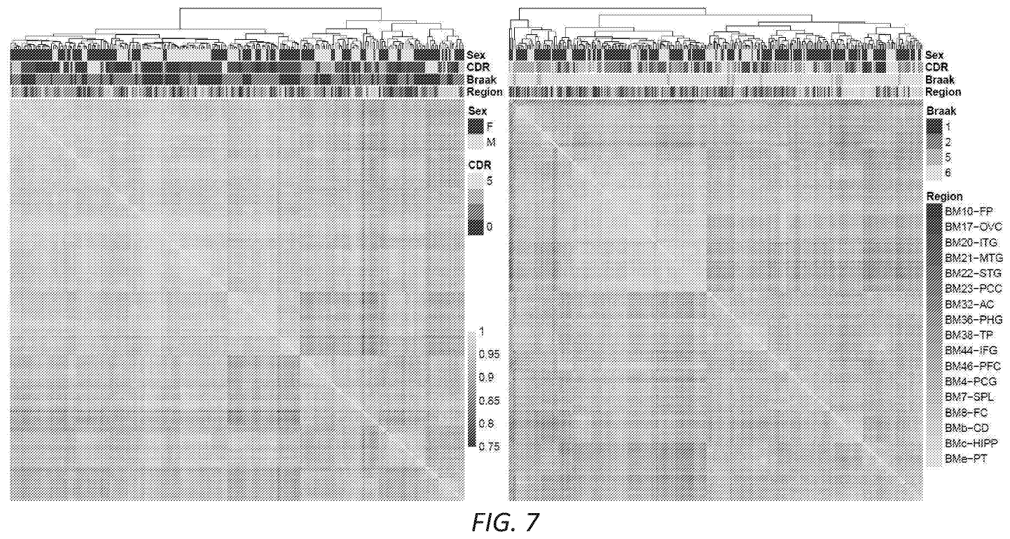

During the ISG-centric analysis of the MSBB dataset, we noted strong correlations in the expression of these genes among groups of samples. We separated the samples into Low-Braak (Braak score=1, 2) and High-Braak (Braak score=5, 6) categories and applied hierarchical clustering to assess the natural grouping of samples within each category. FIG. 7 demonstrates an emerging clustering structure that is not driven by confounders such as gender, brain region, or neuropathological covariates. We postulate that this reveals one dimension of AD heterogeneity, as each cluster of samples exhibits a slightly different, but internally coherent, profile of ISG expression. The implication of these results directly impacts a systematic search of all known drugs that modify interferon signaling (including ruxolitinib and tofacitinib) in early stages (Braak stages 1-2) of AD (left side of FIG. 1) and for AD progression (since both overall and significant subsets of patients show enhanced ISGs).

### REFERENCES

1. Rongvaux A, Jackson R, Harman C C, et al. Apoptotic caspases prevent the induction of type I interferons by mitochondrial DNA. Cell 2014; 159:1563-1577.
2. Liu S, Cai X, Wu J, et al. Phosphorylation of innate immune adaptor proteins MAVS, STING, and TRIF induces IRF3 activation. Science 2015; 347:aaa2630.
3. Jung Y, Dickson D W, Murray M E, et al. TDP-43 in Alzheimer's disease is not associated with clinical FTLD or Parkinsonism. J Neurol 2014; 261:1344-1348.
4. Sakaguchi T, Irie T, Kawabata R, Yoshida A, Maruyama H, Kawakami H. Optineurin with amyotrophic lateral sclerosis-related mutations abrogates inhibition of interferon regulatory factor-3 activation. Neurosci Lett 2011; 505:279-281.
5. Bury J J, Highley J R, Cooper-Knock J, et al. Oligogenic inheritance of optineurin (OPTN) and C9ORF72 mutations in ALS highlights localisation of OPTN in the TDP-43-negative inclusions of C9ORF72-ALS. Neuropathology 2015.
6. Li C, Ji Y, Tang L, et al. Optineurin mutations in patients with sporadic amyotrophic lateral sclerosis in China. Amyotroph Lateral Scler Frontotemporal Degener 2015; 16:485-489.
7. Lagier-Tourenne C, Baughn M, Rigo F, et al. Targeted degradation of sense and antisense C9orf72 RNA foci as therapy for ALS and frontotemporal degeneration. Proc Natl Acad Sci USA 2013; 110:E4530-4539.
8. Mizielinska S, Lashley T, Norona F E, et al. C9orf72 frontotemporal lobar degeneration is characterised by frequent neuronal sense and antisense RNA foci. Acta neuropathologica 2013; 126:845-857.
9. Mizielinska S, Gronke S, Niccoli T, et al. C9orf72 repeat expansions cause neurodegeneration in Drosophila through arginine-rich proteins. Science 2014; 345:1192-1194.
10. Cooper-Knock J, Higginbottom A, Stopford M J, et al. Antisense RNA foci in the motor neurons of C9ORF72-ALS patients are associated with TDP-43 proteinopathy. Acta Neuropathol 2015.
11. DeJesus-Hernandez M, Mackenzie I R, Boeve B F, et al. Expanded GGGGCC hexanucleotide repeat in noncoding region of C9ORF72 causes chromosome 9p-linked FTD and ALS. Neuron 2011; 72:245-256.
12. Renton A E, Majounie E, Waite A, et al. A hexanucleotide repeat expansion in C9ORF72 is the cause of chromosome 9p21-linked ALS-FTD. Neuron 2011; 72:257-268.
13. May S, Homburg D, Schludi M H, et al. C9orf72 FTLD/ALS-associated Gly-Ala dipeptide repeat proteins cause neuronal toxicity and Unc119 sequestration. Acta Neuropathol 2014; 128:485-503.
14. Wen X, Tan W, Westergard T, et al. Antisense proline-arginine RAN dipeptides linked to C9ORF72-ALS/FTD form toxic nuclear aggregates that initiate in vitro and in vivo neuronal death. Neuron 2014; 84:1213-1225.
15. Donnelly C J, Zhang PW, Pham J T, et al. RNA toxicity from the ALS/FTD C9ORF72 expansion is mitigated by antisense intervention. Neuron 2013; 80:415-428.
16. Sareen D, O'Rourke J G, Meera P, et al. Targeting RNA foci in iPSC-derived motor neurons from ALS patients with a C9ORF72 repeat expansion. Sci Transl Med 2013; 5:208ra149.
17. Freibaum B D, Lu Y, Lopez-Gonzalez R, et al. GGGGCC repeat expansion in C9orf72 compromises nucleocytoplasmic transport. Nature 2015; 525:129-133.
18. Chiu I M, Morimoto E T, Goodarzi H, et al. A neurodegeneration-specific gene-expression signature of acutely isolated microglia from an amyotrophic lateral sclerosis mouse model. Cell Rep 2013; 4:385-401.
19. Frakes A E, Ferraiuolo L, Haidet-Phillips A M, et al. Microglia induce motor neuron death via the classical NF-kappaB pathway in amyotrophic lateral sclerosis. Neuron 2014; 81:1009-1023.
20. Mukherjee P, Woods T A, Moore R A, Peterson K E. Activation of the innate signaling molecule MAVS by bunyavirus infection upregulates the adaptor protein SARM1, leading to neuronal death. Immunity 2013; 38:705-716.
21. Mukherjee P, Winkler C W, Taylor K G, et al. SARM1, Not MyD88, Mediates TLR7/TLR9-Induced Apoptosis in Neurons. J Immunol 2015; 195:4913-4921.
22. Scheuner D, Gromeier M, Davies M V, et al. The double-stranded RNA-activated protein kinase mediates viral-induced encephalitis. Virology 2003; 317:263-274.
23. Baruch K, Deczkowska A, David E, et al. Aging-induced type I interferon response at the choroid plexus negatively affects brain function. Science 2014; 346:89-93.
24. Zhu P J, Huang W, Kalikulov D, et al. Suppression of PKR promotes network excitability and enhanced cognition by interferon-gamma-mediated disinhibition. Cell 2011; 147:1384-1396.
25. Bonin M, Oberstrass J, Lukacs N, et al. Determination of preferential binding sites for anti-dsRNA antibodies on double-stranded RNA by scanning force microscopy. RNA 2000; 6:563-570.
26. Gendron T F, van Blitterswijk M, Bieniek K F, et al. Cerebellar c9RAN proteins associate with clinical and neuropathological characteristics of C9ORF72 repeat expansion carriers. Acta Neuropathol 2015; 130:559-573.
27. Donato R, Miljan E A, Hines S J, et al. Differential development of neuronal physiological responsiveness in two human neural stem cell lines. BMC Neurosci 2007; 8:36.
28. Kim Y H, Choi S H, D'Avanzo C, et al. A 3D human neural cell culture system for modeling Alzheimer's disease. Nat Protoc 2015; 10:985-1006.
29. Feldman R I, Wu J M, Polokoff M A, et al. Novel small molecule inhibitors of 3-phosphoinositide-dependent kinase-1. J Biol Chem 2005; 280:19867-19874.
30. Tu D, Zhu Z, Zhou A Y, et al. Structure and ubiquitination-dependent activation of TANK-binding kinase 1. Cell Rep 2013; 3:747-758.
31. Li W, Jin Y, Prazak L, Hammell M, Dubnau J. Transposable elements in TDP-43-mediated neurodegenerative disorders. PloS one 2012; 7:e44099.
32. Saldi T K, Ash P E, Wilson G, et al. TDP-1, the Caenorhabditis elegans ortholog of TDP-43, limits the accumulation of double-stranded RNA. The EMBO journal 2014; 33:2947-2966.
33. Polymenidou M, Lagier-Tourenne C, Hutt K R, et al. Long pre-mRNA depletion and RNA missplicing contribute to neuronal vulnerability from loss of TDP-43. Nature neuroscience 2011; 14:459-468.
34. Amit I, Garber M, Chevrier N, et al. Unbiased reconstruction of a mammalian transcriptional network mediating pathogen responses. Science 2009; 326:257-263.
35. Douville R, Liu J, Rothstein J, Nath A. Identification of active loci of a human endogenous retrovirus in neurons of patients with amyotrophic lateral sclerosis. Annals of neurology 2011; 69:141-151.
36. Saldi T K, Ash P E, Wilson G, et al. TDP-1, the Caenorhabditis elegans ortholog of TDP-43, limits the accumulation of double-stranded RNA2014.
37. Li W, Lee M H, Henderson L, et al. Human endogenous retrovirus-K contributes to motor neuron disease. Sci Transl Med 2015; 7:307ra153.
38. McAllister C S, Samuel C E. The RNA-activated protein kinase enhances the induction of interferon-beta and apoptosis mediated by cytoplasmic RNA sensors. The Journal of biological chemistry 2009; 284:1644-1651.
39. Gitlin L, Barchet W, Gilfillan S, et al. Essential role of mda-5 in type I IFN responses to polyriboinosinic:polyribocytidylic acid and encephalomyocarditis picornavirus. Proceedings of the National Academy of Sciences of the United States of America 2006; 103:8459-8464.
40. Talkowski M E, Rosenfeld J A, Blumenthal I, et al. Sequencing chromosomal abnormalities reveals neurodevelopmental loci that confer risk across diagnostic boundaries. Cell 2012; 149:525-537.
41. Reilly M T, Faulkner G J, Dubnau J, Ponomarev I, Gage F H. The role of transposable elements in health and diseases of the central nervous system. The Journal of neuroscience: the official journal of the Society for Neuroscience 2013; 33:17577-17586.
42. Ling J P, Pletnikova O, Troncoso J C, Wong P C. NEURODEGENERATION. TDP-43 repression of nonconserved cryptic exons is compromised in ALS-FTD. Science 2015; 349:650-655.
43. Liddicoat B J, Piskol R, Chalk A M, et al. RNA editing by ADAR1 prevents MDAS sensing of endogenous dsRNA as nonself. Science 2015; 349:1115-1120.
44. Rao J S, Kellom M, Kim H W, Rapoport S I, Reese E A. Neuroinflammation and synaptic loss. Neurochemical research 2012; 37:903-910.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. (canceled)

2. (canceled)

3. (canceled)

4. (canceled)

5. A method of treating a neurodegenerative disease in a subject in need thereof, the method comprising administering to the subject a therapeutically effective amount of one or more inhibitors of TANK Binding Kinase 1 (TBK1), I kappa B kinase (IKK), Signal Transducer and Activator of Transcription 1 (STAT1), and/or Janus Kinase 1 and/or Janus Kinase 2 (Jak1/2), thereby treating the neurodegenerative disease in the subject.

6. The method of claim 5, wherein the inhibitor is selected from the group consisting of:
a small molecule inhibitor of a TBK1, IKK, STAT1, or Jak1/2 protein;
an inhibitory nucleic acid that targets a TBK1, IKK, STAT1, or Jak1/2 transcript; and/or
an antibody that binds to and inhibits a TBK1, IKK, STAT1, or Jak1/2 protein.

7. The method of claim 6, wherein the small molecule inhibitor is selected from the group consisting of BX-795; TPCA-1; Ruxolitinib; fludarabine; amlexanox, Pacritinib-1, XL019, Filgotinib, AZD1480, Baricitinib, Tofacitinib, IKK16, momelotinib, and TG101348.

8. The method of claim 5, wherein the neurodegenerative disease is Alzheimer's disease, amyotrophic lateral sclerosis, frontotemporal dementia, Cockayne Syndrome (CS), Xeroderma Pigmentosum (XP), Trichothiodystrophy (TTD), Ataxia with Occulomotor Apraxia-1 (AOA1), Spinocerebellar Ataxia with Axonal Neuropathy (SCAN1), Ataxia Telangiectasia (A-T) or A-T Like Disease (ATLD), ATR-Seckel Syndrome, Nijmegen Breakage Syndrome (NBS), LIG4 Syndrome, Aicardi-Goutier's syndrome and related interferonopathies, Down's Syndrome, or XLF Syndrome.

9. The method of claim 8, wherein the amyotrophic lateral sclerosis (ALS) is C9orf72-linked ALS, fused in sarcoma (FUS)-linked ALS, TAR DNA-binding protein 43 (TDP-43)-linked ALS, C9orf72-linked frontotemporal dementia (FTD), FUS-linked FTD, and TDP-43-linked FTD, C9orf72-linked AD, or sporadic ALS.
